(19) United States
(12) Patent Application Publication
(10) Pub. No.: US 2004/0236333 A1
(43) Pub. Date: Nov. 25, 2004

(54) Uniplate cervical device

(76) Inventor: Paul S. Lin, Lewisburg, PA (US)

Correspondence Address: CALFEE HALTER & GRISWOLD, LLP, 800 SUPERIOR AVENUE, SUITE 1400, CLEVELAND, OH, 44114, US

(21) Appl. No.: 10/804,743

(22) Filed: Mar. 19, 2004

Related U.S. Application Data
(60) Provisional application No. 60/456,787, filed on Mar. 21, 2003.

(51) Int. Cl. A61B 17/56

(52) U.S. Cl. 606/69

(57) ABSTRACT

A plate system used to maintain a generally fixed distance between vertebrae during the surgical repair of damaged vertebrae and/or the discs therebetween. The plate system is provided for use in conjunction with a cervical implant or alone. The system includes a plate which may be held in place with screws and nuts. The plate defines multiple apertures for the passage of the screws, one of which is a slot and as a result a single sized plate may be used for many different situations where the gap between adjacent vertebrae is varied. The plate also includes at least one cleat which provides and additional grip between the plate and vertebrae.

### CROSS-REFERENCE TO RELATED APPLICATIONS

This application claims priority to U.S. Provisional Application Ser. No. 60/456,787 filed on Mar. 21, 2003, currently pending, and incorporates its entirety herein.

### FIELD OF THE INVENTION

This invention generally relates to prostheses for the spine and more specifically to a plate system to separate adjacent cervical vertebrae.

### BACKGROUND OF THE INVENTION

The spine is the central support column for the human body. The cervical region of the spine is located in the area of the neck and is comprised of cervical vertebrae separated by disks. A diseased spine suffers from deterioration of a vertebra, disk or both. Disks and vertebrae may also be damaged by physical causes as well. Surgical repair consists of fusing adjacent vertebrae together by means of a bone graft. It is necessary to keep the adjacent vertebrae spaced at a certain distance at the time the bone graft is growing and fusing the adjacent vertebrae together. Traditionally, this has been done mechanically. Typically, the systems are comprised of plates and screws or rods and screws.

A problem with prior art mechanical devices of the plate and screw type is their lack of adjustability. A surgeon does not know until an incision is made and screws are set what the desired gap size between adjacent vertebrae should be, thus, a large inventory of plates must be kept. Some systems such as that described by U.S. Pat. No. 5,904,683 allow for some adjustment by tilting the set screws. However, a system which allows for a large amount of plate adjustment is desired so a doctor's inventory of plates may be kept small.

Another problem with the prior art plate systems is that too many screws or too small of screws are used. Prior art which relies upon four or more screws has problems when part of one of the vertebra being screwed into is diseased or fractured. Alternatively, smaller screws have a problem with backing out of their set positions over time. What is desired is a minimum number of large screws which may be placed in multiple positions.

Another problem with the prior art is that most plate inventions are not designed to work in tandem with implant devices. The result is a problem with the size to strength ratio of plate or rod or plate systems. Because they are designed to be used individually they often must be of a large size in order to guarantee separation of adjacent vertebrae. A drawback to these systems is that larger incisions must be made in order to install the systems and more bone screws must be used to secure the systems in place. Larger incisions and increased dissection lead to greater possibilities for dysphagia and/or recurrent nerve paralysis. What is desired is a plate system of reduced size which may be used in conjunction with other support structures such as cage or biscuit type implants.

### SUMMARY OF THE INVENTION

The present invention overcomes disadvantages in the prior art by providing an improved cervical system and method of holding spinal vertebrae apart. The system includes a cervical plate which is smaller and more universal than known plates. The plate may be used alone, but is preferably used in combination with known cervical implants, typically of a biscuit or cage type. The plate of the system also includes at least one cleat, which allows the number of screws used to hold the plate in position to be reduced and their sized increased.

The invention provides in one aspect a plate for holding two vertebrae apart. The plate has at least two apertures, one being a slot. The plate includes interior and exterior faces and the cleat or cleats protrude from the interior face. Cancellous bone screws may be used to attach the plate to the vertebrae. The apertures within the plate pass over the trailing end of the screws and the plate secured using nuts which threadedly attach to the screws.

The invention additionally provides a central beam integral within the plate which is aligned with the center of the spine when the plate is installed. This beam is continuous, avoiding any of the apertures defined by the plate, and provides superior strength where it is needed most.

The invention, in the aspects described above, provides the advantage of a physical support device which prevents the collapse of adjacent vertebrae in the event a bone graft is unsuccessful. Further the invention provides a plate having superior strength which may be used for a wide variety of gap sizes between adjacent vertebrae. These and other features, aspects and advantages of the present invention will be fully described by the following description, appended claims, and accompanying drawings.

### BRIEF DESCRIPTION OF THE FIGURES

FIG. 1a is a side view of a uniplate cervical system attached to spinal vertebrae;

FIG. 1b is a front view of a uniplate cervical system attached to spinal vertebrae;

FIG. 1c is a side view of a uniplate cervical system attached to spinal vertebrae used in conjunction with a cervical implant;

FIG. 2a is a side view of a cancellous bone screw;

FIG. 2b is an end view of the cancellous bone screw;

FIG. 2c is a cross sectional view of the cancellous bone screw;

FIG. 2d is a cross sectional detail view of a portion of the screw of FIG. 2c;

FIG. 3 is a side view of a distracter;

FIG. 4a is a top view of a cervical plate;

FIG. 4b is a cross sectional view of one portion of the cervical plate;

FIG. 4c is a cross sectional view of another portion of the cervical plate;

FIG. 4d is a side view of the cervical plate;

FIG. 4e is a perspective view of the cervical plate;

FIG. 4f is isolated view of a cleat from the cervical plate;

FIG. 5a is a top view of a cervical plate with multiple cleats;

FIG. 5b is a cross sectional view of one portion of the cervical plate of FIG. 5a;

FIG. 5c is a cross sectional view of another portion of the cervical plate of FIG. 5a;

FIG. 5d is a side view of the cervical plate of FIG. 5a;

FIG. 5e is a perspective view of the cervical plate of FIG. 5a;

FIG. 5f is an isolated view of a cleat from a cervical plate of FIG. 5a;

FIG. 6a is a side view of a locking nut;

FIG. 6b is a top view of the locking nut; and

FIG. 6c is a perspective view of the locking nut.

### DETAILED DESCRIPTION OF THE INVENTION

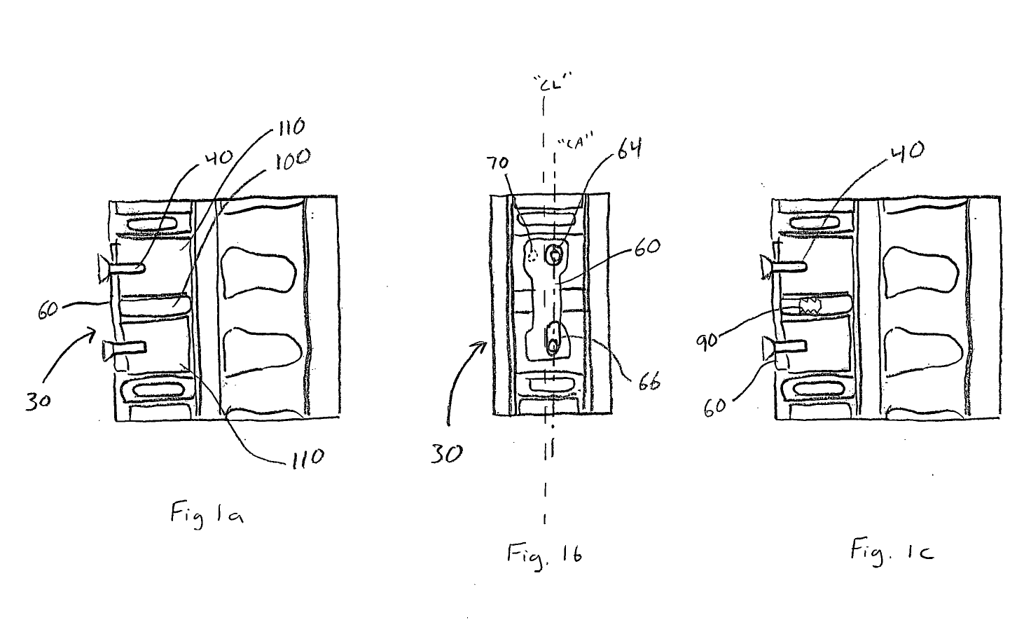

Referring to the drawings, FIGS. 1a and 1b illustrate a preferred uniplate cervical system 30, as will be described in greater detail below, which includes two cancellous threaded bone screws 40, a cervical plate 60 with at least one stabilizing cleat or projection 70 (a stabilizing cleat can be any bump, spike or other protuberance that furnishes a grip), and mounting apertures 64 and 66, one of which may be a slot. FIG. 1c shows an alternate embodiment of a uniplate cervical system which additionally includes a cervical implant 90, comprising a cage or biscuit device to keep adjacent vertebrae spaced consistently.

As schematically represented in FIG. 1a, two cancellous threaded bone screws 40 are used to attach the system 30 to damaged vertebrae 110, or vertebrae between which a disc 100 has been damaged. A cancellous screw is any type of fastener in which the threads are designed to penetrate a soft substrate as opposed to a hard substrate, such as metal.

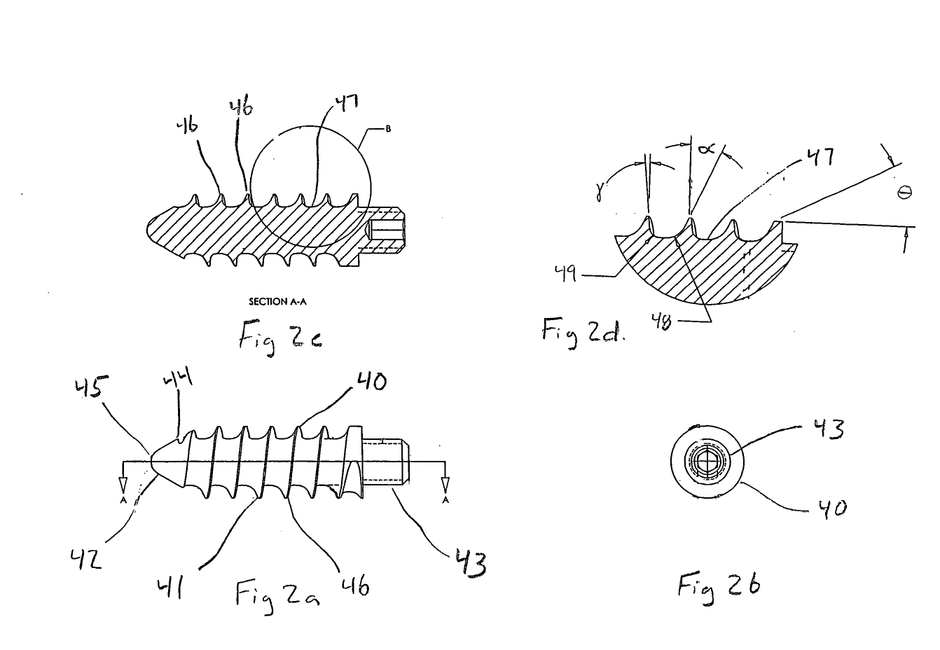

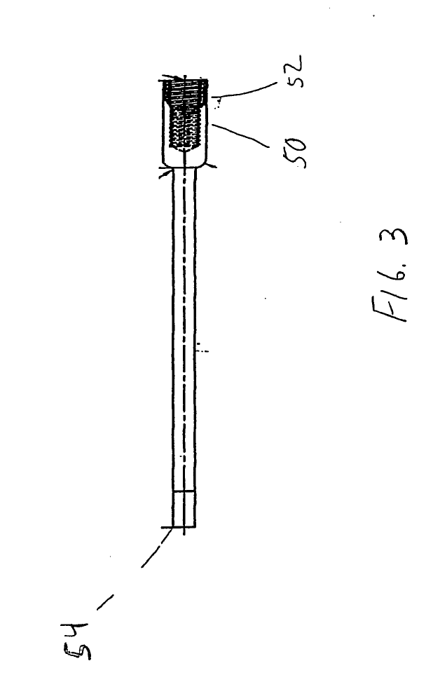

As shown in FIG. 2a each bone screw 40 has a leading end 42 and trailing end 43. Preferably the bone screw 40 has a length in a range from about 8 to about 30 millimeters omitting the trailing end 43. The trailing end 43 has a different surface configuration (threads, etc.) than the remainder of the bone screw 40. The first turn 44 of the thread at the leading end 42 of the bone screw 40 may have a tapered crest which prevents the bone screw 40 from backing out of its set position after being screwed into the bone. The leading end 42 of the bone screw 40 may have a cone point 45 which is rounded at the tip. A main body section 41 of the bone screw includes threads 46. The surface of trailing end 43 of the bone screw 40 may also be threaded on the exterior with a thread (different from the main body thread) which allows a distracter adapter 50 shown in FIG. 3 to be attached to the bone screw 40 easily. Alternatively, the surface of the trailing end 43 could include flats which allow a clamp to be applied for attachment. Referring to FIG. 2b, the interior of the trailing end 43 of the bone screw 40 may also be machined allowing the bone screw 40 to be driven into place with a tool. The interior of the bone screw 40 may be machined leaving a void with hexagonal sides. However, square sides or internal threads may also be used for connection to a driving tool.

The cancellous bone screw 40 may be made of a titanium alloy, preferably Ti-6AI-4V, but may be any material possessing superior strength and which is compatible with the human body. Referring to FIG. 2a, the bone screw 40 may be also of any size and of any known type that is suitable for use in securing bone material. Preferably, however, the bone screws have a main body 41 minor diameter not exceeding 3.5 millimeters.

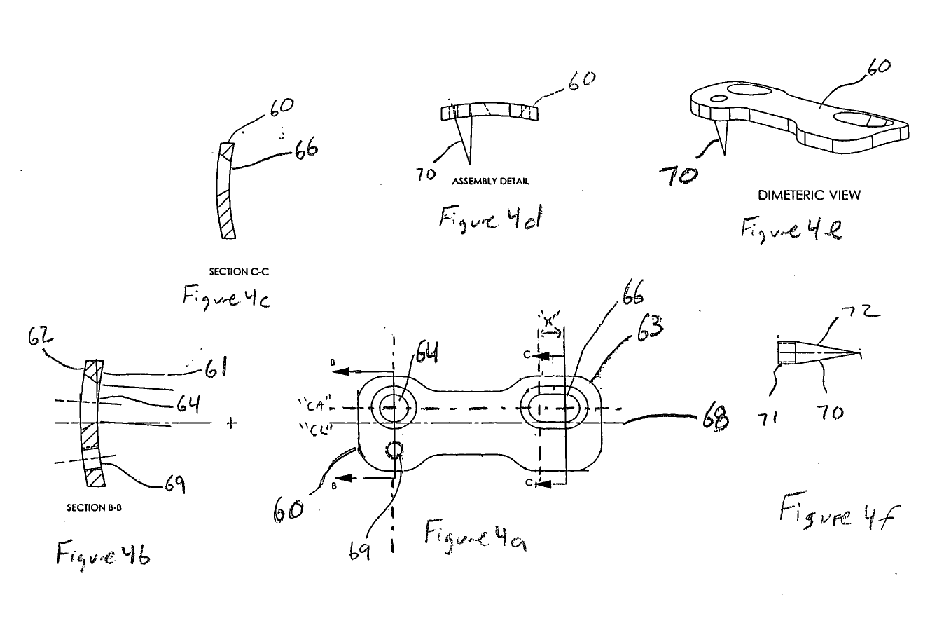

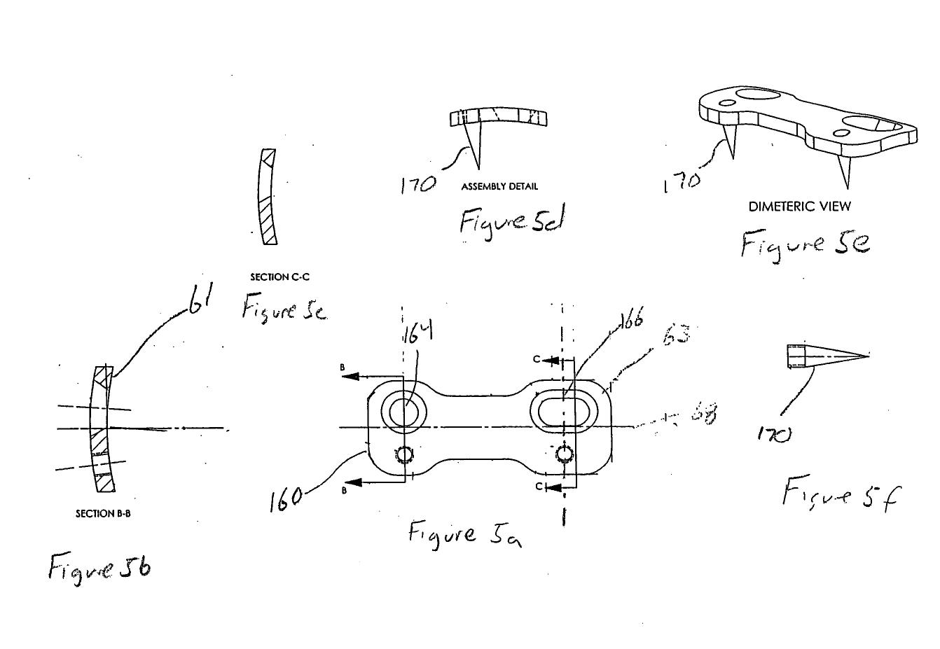

The uniplate cervical system 30 further comprises a cervical plate 60 as shown in FIGS. 1a-c, and 4a-e which holds two vertebrae apart at a desired distance. FIGS. 5a-e show an alternate embodiment of the cervical plate 160 having a different size and including multiple cleats 170. Referring to FIGS. 4a-e, the cervical plate 60 is shown as a generally rectangular shaped piece having an interior side 61 and an exterior side 62, rounded edges 63 and being bowed along its width, but may be other shapes. Preferably, the bow of the cervical plate 60 is defined by an angled radius of curvature of about 27 millimeters. The thickness of the cervical plate 60 is preferably about 1.6 millimeters

The cervical plate 60 defines two mounting apertures 64 and 66 through which the trailing ends 43 of bone screws 40 pass when the uniplate cervical system 30 is installed. Round aperture 64 is substantially the same size as the diameter of the trailing end 43 of the bone screw 40 such that there is no substantial play in the fitting between the bone screw 40 and the cervical plate 60. The round aperture 64 may be countersunk which improves seating of a locking nut 80 further reducing any chance for movement of the cervical uniplate system 30 when in place and secured. The round aperture 64 is preferably countersunk at about 66 degrees. The round aperture 64 is preferably offset, at its center, from a centerline “CL” passing from a leading edge to a trailing edge of the cervical plate 60. The cervical plate 60 also defines an aperture 69 for the mounting of a cleat 70. This aperture 69 may or may not be threaded. The cleat aperture 69 is preferably located along the same transverse axis as the center of the round aperture 64. The cleat aperture 69 is preferably offset from the cervical plate centerline “CL”.

The aperture 66 is a slot having a predetermined length, a width substantially the same size as the diameter of the trailing edge 43 of the bone screw 40 and rounded ends with a radius substantially the same size as the radius of the trailing edge 43 of the bone screw 40. The perimeter of the aperture 66 may be countersunk to allow improved seating of a locking nut 80. The slotted cervical plate 60 allows a surgeon to address multiple situations, where the desired gap between vertebra varies, while carrying a small inventory of plates. Further benefit is derived from the small amount of vertebral settling that the aperture 66 allows. Should settling occur, the screw 40 will slide in the aperture 66 within the plate 60, rather than pull out from the vertebra. The aperture 66 may be of any appropriate length, but preferably has a length of 2.8 millimeters (see designation “x”) from the radial centers of each rounded edge. Alternatively, aperture 66 may be a circular aperture similar to aperture 64.

In use, the apertures 64 and 66 are placed along a common axis (“CA”) which is parallel to, but offset from the central axis of the patient's spine as shown in FIG. 1b. Thus, when mounted, the beam 68 (co-linear with centerline “CL”) inherent in the cervical plate 60 which is also co-linear with the spine's central axis, is devoid of any holes or formations, resulting in a region of superior strength where it is most desired. Placement of the bone screws 40 and cervical plate apertures 64 and 66 may be on either side of the spine's central axis, thus giving a surgeon greater flexibility to avoid attachment to weak or deteriorated bone.

Referring back to FIGS. 4a-e, the size of the cervical plate 60 may be of any appropriate size required to perform its function. However, preferably the size of the cervical plate 60 from leading edge to trailing edge may be within a range of about 28 millimeters to about 43 millimeters. The width of the cervical plate 60 preferably is between 10 and 14 millimeters. The portion of reduced width in the central region of the cervical plate preferably between 6 and 10 millimeters. The nominal size of the cervical plate 60 is measured from the center of the round aperture 64 to the center of the aperture 66 and may be in a range from about 18 to about 32 millimeters.

One cleat 70 protrudes from the interior side 61 of the cervical plate 60 and may be embedded within a vertebra when the system 30 is installed. Referring to FIG. 4f, each cleat 70 preferably tapers to a sharp point from its anterior to its posterior end. Alternatively, the cleat 70 may be a tapering rectangular solid or any other shape that can grip a structure comprised of bone. The cervical plate 60 is preferably made of titanium alloy (Ti-6AL-4V), but may be made of any material possessing superior strength which is compatible with the human body. The cervical plate 60 can be passivated and given a #10 glass bead finish. The cleat 70 is manufactured out of any common material used in the bone grafting art. Cleat 70 is usually a separate piece that is threaded into the cervical plate 60, but may also simply be press fit into cervical plate 60.

An alternate embodiment of the invention, cervical plate 160 is shown in FIGS. 5a-e and includes multiple cleats 170. This plate 160 also defines multiple apertures 164 and 166 for the passage of mounting screws. The second cleat 170 is located with its center along a transverse axis which passes through the center of the aperture 166.

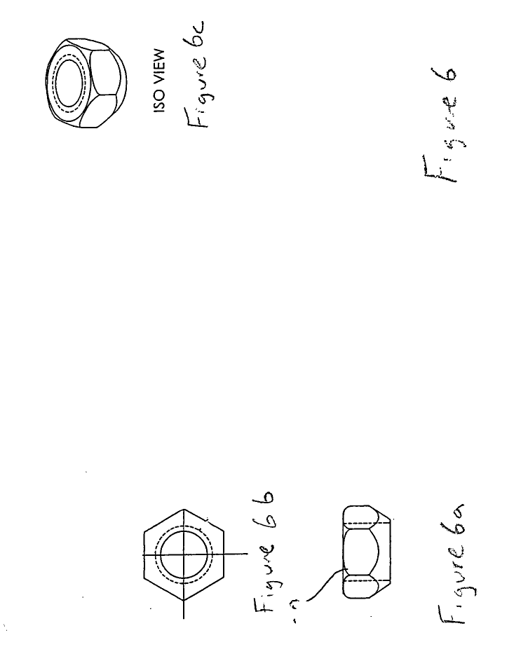

Locking nuts 80 are known and screwed onto the trailing ends 43 of the cancellous bone screws 40 and act to lock the cervical plate 60 or 160 in place. Referring to FIGS. 6a-c a locking nut 80 may be hexagonal shaped with a chamfered end allowing superior engagement with the countersunk area of the cervical plate 60 thus disallowing relative movement. The nut 80 may be made of titanium alloy, but may be made of any strong material which is compatible with the human body. Locking nuts 80 may be of the type sold by a company called Spiralock Corporation located in Madison Heights, Mich.

Referring to FIGS. 1a and 1b, the uniplate cervical system 30 is shown being attached to two vertebrae in the cervical section of the human spine. The spine may be accessed anteriorally. A limited discectomy is performed to identify the orientation of the space between the two mounting vertebrae. In no preferred order, prior to insertion of the plate, a single cancellous bone screw 40 is screwed into the vertebra adjacent to the altered disc and a second bone screw 40 is attached to the second adjacent vertebra. The screws 40 are offset from the central axis of the spine, but are placed such that their centers are on a line substantially parallel to the central axis. A distraction adapter 50 screws onto the machine thread or clamps to each bone screw 40. The adapter 50 may be any tool which has an end to mate with the cancellous bone screw 40 and is of a sufficient length to extend outside of the body. Distracter adapter 50, shown in FIG. 3 may have an internal female machine thread 52 at one end, although this may also be a hexagonal shaped adapter or a clamp. Distracter adapter 50 also has a machined opposite end, 54 which may be in a hexagonal shape, so as to allow connection to a distracter tool. A distracter tool, such as a Caspar distracter, is connected to each distracter adapter 50. A surgeon applies force to the distracter tool to separate the vertebrae apart. The use of cancellous threaded screws 40 allows for more force to be applied through the distracter tool than was possible with prior art.

A partial or complete discectomy is performed and bone graft material is placed between the spaced vertebrae. Additionally or alternatively, an anterior weight-sharing device or cervical implant may be placed between the spaced vertebrae. A typical cervical implant is shown in U.S. Pat. No. 6,635,086 to Lin or U.S. Pat. No. 6,193,755 to Metz-Stavenhagen et al. Distracter tool and distracter adapters 50 are removed from the cancellous screws 40 allowing cervical plate 60 to be put in place. The appropriate sized cervical plate 60 is chosen after the distance between the cancellous screws 40 is measured. A benefit of having aperture 66 be a slot upon the cervical plate 60 is that one plate 60 can be used for a variety of distances between the cancellous screws 40. Thus, a smaller inventory of cervical plates 60 is required in the surgeon's inventory. The trailing ends 43 of the two cancellous bone screws 40 protrude through the mounting apertures 64 and 66 of the cervical plate 60 when it is in position. Chamfered nuts 80 are applied to the ends of the cancellous screws 40 to lock the cervical plate 60 in place.

Due to the presence of cleat 70 or 170, the plate may be placed and secured to the vertebrae without any additional screws being inserted into the vertebrae. This results in a much faster and streamlined medical procedure and also results in fewer, larger holes that need be created in bone tissue that usually is already damaged in some way.

Although the invention has been shown and described with reference to certain preferred and alternate embodiments, the invention is not limited to these specific embodiments. Minor variations and insubstantial differences in the various combinations of materials and methods of application may occur to those of ordinary skill in the art while remaining within the scope of the invention as claimed and equivalents. Use of the term “or” herein is the inclusive, and not the exclusive use. The preferred embodiments described above are not in any way intended to limit the invention as claimed below.

1. A plate attachable to two adjacent spinal vertebrae comprising:
first and second apertures defined by the plate for the passage of screws which attach to the spinal vertebrae; and
a cleat extending from an interior face of said plate.

2. The plate of claim 1 in which one of said first and second apertures is a slot.

3. The plate of claim 1 including a central portion having a reduced width.

4. The plate of claim 1 wherein said cleat is aligned along a common transverse axis with the center of said first aperture.

5. The plate of claim 1 including a second cleat wherein said second cleat is aligned along a common transverse axis with the center of said second aperture.

6. The plate of claim 1 wherein the plate is bowed along its width.

7. A plate system for holding two vertebrae apart comprising:
first and second screws for attaching to said vertebrae;
a plate having interior and exterior faces;
said plate defining at least two apertures;
said interior face being provided with a cleat.

8. The system of claim 7 wherein said screws are of a cancellous type.

9. The system of claim 7 wherein said plate has a length not exceeding 43 millimeters and a width not exceeding 12 millimeters.

10. The system of claim 7 wherein said cleat is in the form of a conical spike.

11. The system of claim 7 wherein said plate further includes a leading edge and a trailing edge and a beam integrally formed within said plate, said beam extending from said leading edge to trailing edge in an unbroken line generally along a centerline of said plate.

12. A plate system for holding two vertebrae apart comprising:
a plate attachable to the two vertebrae;
first and second apertures defined by said plate for the passage of screws which attach to the vertebrae wherein at least one of said apertures is a slot;
a single cleat extending from an interior face of said plate; and
a cervical implant to be placed between the spinal vertebrae.

13. The plate system of claim 12 wherein said plate includes a second cleat.

14. The plate system of claim 13 wherein the first cleat is aligned along a common traverse axis with the center of the first aperture and the second cleat is aligned along a common transverse axis with the center of said second aperture.

15. The plate system of claim 12 wherein said plate further includes a leading edge and a trailing edge and a beam integrally formed within said plate, said beam extending from said leading edge to trailing edge in an unbroken line generally along a centerline of said plate and said plate is bowed along its width.

16. The plate system of claim 12 further including first and second screws for attaching to said vertebrae and first and second nuts attachable to said first and second screws respectively, said first and second nuts for maintaining said plate in a position to hold said vertebrae apart.

17. A method of holding two spinal vertebrae apart comprising the steps of:
a. placing a single screw into each of two adjacent vertebrae of a human spine having a central axis,
b. attaching a distraction adapter to each screw,
c. attaching the distraction adapters to a distraction tool,
d. spreading said vertebrae by actuation of said distraction tool,
e. mounting a plate upon the screws, said plate having first and second apertures defined by the plate for the passage of the screws wherein at least one of said apertures is a slot; and
f. securing said plate to said screws with two fasteners.

18. The method of claim 16 further including the step of placing an implant between said two adjacent vertebrae.
